(19) United States
(12) Patent Application Publication
(10) Pub. No.: US 2018/0311244 A1
(43) Pub. Date: Nov. 1, 2018

(54) METHODS OF TREATING LIVER DISEASE

(71) Applicant: Gilead Sciences, Inc., Foster City, CA (US)

(72) Inventors: Jamie Geier Bates, Burlingame, CA (US); David Gordon Clarkson Breckenridge, San Mateo, CA (US); Grant Raymond Budas, Palo Alto, CA (US)

(21) Appl. No.: 15/937,678

(22) Filed: Mar. 27, 2018

Related U.S. Application Data
(60) Provisional application No. 62/513,311, filed on May 31, 2017.
(60) Provisional application No. 62/511,027, filed on May 25, 2017.
(60) Provisional application No. 62/482,097, filed on Apr. 5, 2017.
(60) Provisional application No. 62/477,859, filed on Mar. 28, 2017.

(51) Int. Cl. A61K 31/519; A61K 31/4439; A61P 1/16

(52) U.S. Cl. CPC A61K 31/519; A61P 1/16; A61K 31/4439

(57) ABSTRACT

The present disclosure relates to a method of preventing and/or treating liver disease comprising administering an ASK1 inhibitor in combination with a ACC inhibitor to a patient in need thereof.

### CROSS-REFERENCE TO RELATED APPLICATION

This application claims the benefit under 35 U.S.C. § 119(e) of United States Provisional Application Nos. 62/477,859, filed Mar. 28, 2017, 62/482,097, filed Apr. 5, 2017, 62/511,027, filed May 25, 2017, and 62/513,311, filed May 31, 2017, each of which are hereby incorporated by reference in their entirety.

### FIELD

The present disclosure relates to methods of preventing and/or treating liver diseases.

### BACKGROUND

Liver disease is a leading cause of death worldwide. Liver disease may be caused by infection, injury, exposure to drugs or toxic compounds, alcohol, impurities in foods, the abnormal build-up of normal substances in the blood, an autoimmune process, a genetic defect (such as haemochromatosis), or unknown cause(s). Liver disease is generally classified as acute or chronic based upon the duration of the disease.

More than 20 percent of the population has non-alcoholic fatty liver disease (NAFLD) according to the American Liver Foundation. When left untreated, NAFLD can progess to non-alcoholic steatohepatitis (NASH) causing serious adverse effects. An estimated 16 million adults in the United States have NASH and approximately 50% have advanced hepatic fibrosis (bridging fibrosis or cirrhosis) associated with NASH. Based on these numbers NASH is expected to become the leading indication for liver transplantation by 2020. NASH is characterized by the presence of steatosis and by other features including hepatocellular degeneration (ballooning, Mallory hyaline), inflammatory cell infiltration and development of progressive fibrosis.

There are no currently approved therapies for the treatment of NASH and no therapies that reduce fibrosis and/or steatosis in patients with NASH. Accordingly, there remains a need to provide new effective pharmaceutical agents to treat liver disease or the symptoms of liver disease.

### SUMMARY

Disclosed herein is a method of treating and/or preventing liver disease in a patient in need thereof, comprising administering to the patient a therapeutically effective amount of an apoptosis signal regulating kinase 1 (ASK1) inhibitor in combination with a therapeutically effective amount of Acetyl-CoA Carboxylase (ACC) inhibitor. The liver disease can be any liver disease, including, but not limited to, chronic and/or metabolic liver diseases, nonalcoholic fatty liver disease (NAFLD), and nonalcoholic steatohepatitis (NASH).

In certain embodiments, provided herein is a method of treating and/or preventing nonalcoholic steatohepatitis (NASH) in a patient in need thereof, comprising administering to the patient a therapeutically effective amount of an ASK1 inhibitor in combination with a therapeutically effective amount of an ACC inhibitor.

In the methods provided herein, the ASK1 inhibitor and the ACC inhibitor can be coadministered. In such embodiments, the ASK1 inhibitor and the ACC inhibitor can be administered together as a single pharmaceutical composition, or separately in more than one pharmaceutical composition. Accordingly, also provided herein is a pharmaceutical composition comprising a therapeutically effective amount of an ASK1 inhibitor and a therapeutically effective amount of an ACC inhibitor.

### DESCRIPTION OF THE DRAWINGS

FIG. 1. Macrovesicular steatosis as % macrovesicular area. (*p<0.05; **p<0.01; ***p<0.001, ****p<0.0001 significantly different from vehicle by ANOVA; # significantly different from either single agent). Graph shows mean±SEM.

FIG. 2. Liver Triglycerides in umol/g. (*p<0.05; **p<0.01; ***p<0.001, ****p<0.0001 significantly different from vehicle by ANOVA; # significantly different from either single agent by t-test). Graph shows mean±SEM.

FIG. 3. Liver cholesterol in mg/g. (*p<0.05; **p<0.01; ***p<0.001, ****p<0.0001 significantly different from vehicle by ANOVA; # significantly different from either single agent by t-test). Graph shows mean±SEM.

FIG. 4. ALT IU/L. (*p<0.05; **p<0.01; ***p<0.001, ****p<0.0001 significantly different from vehicle by ANOVA; # significantly different from either single agent by t-test). Graph shows mean±SEM.

FIG. 5. Hepatic expression of liver fibrosis genes Timp1 measured by quantitative RT-PCR. (*p<0.05; **p<0.01; ***p<0.001, ****p<0.0001 significantly different from vehicle by ANOVA; # significantly different from either single agent by t-test). Graph shows mean±SEM.

FIG. 6. Hepatic expression of liver fibrosis genes Colla1 measured by quantitative RT-PCR. (*p<0.05; **p<0.01; ***p<0.001, ****p<0.0001 significantly different from vehicle by ANOVA; # significantly different from either single agent by t-test). Graph shows mean±SEM.

FIG. 7. Percent PSR Area. (*p<0.05; **p<0.01; ***p<0.001, ****p<0.0001 significantly different from vehicle by ANOVA; # significantly different from either single agent by t-test). Graph shows mean±SEM.

FIG. 8. Plasma TIMP1 ng/mL (*p<0.05; **p<0.01; ***p<0.001, ****p<0.0001 significantly different from vehicle by ANOVA; # significantly different from either single agent by t-test). Graph shows mean±SEM.

FIG. 9. Plasma HA ng/ml (*p<0.05; **p<0.01; ***p<0.001, ****p<0.0001 significantly different from vehicle by ANOVA; # significantly different from either single agent by t-test). Graph shows mean±SEM.

### DETAILED DESCRIPTION

### Definitions and General Parameters

As used in the present specification, the following terms and phrases are generally intended to have the meanings as set forth below, except to the extent that the context in which they are used indicates otherwise.

As used herein, the term “about” used in the context of quantitative measurements means the indicated amount ±10%, or alternatively the indicated amount ±5% or ±1%.

The term “pharmaceutically acceptable salt” refers to a salt of a compound disclosed herein that retains the biological effectiveness and properties of the underlying compound, and which is not biologically or otherwise undesirable. There are acid addition salts and base addition salts. Pharmaceutically acceptable acid addition salts may be prepared from inorganic and organic acids. Acids and bases useful for reaction with an underlying compound to form pharmaceutically acceptable salts (acid addition or base addition salts respectively) are known to one of skill in the art. If the compounds described herein are obtained as an acid addition salt, the free base can be obtained by basifying a solution of the acid salt. Conversely, if the product is a free base, an addition salt, particularly a pharmaceutically acceptable addition salt, may be produced by dissolving the free base in a suitable organic solvent and treating the solution with an acid, in accordance with conventional procedures for preparing acid addition salts from base compounds. Those skilled in the art will recognize various synthetic methodologies that may be used to prepare nontoxic pharmaceutically acceptable addition salts. Pharmaceutically acceptable acid addition salts may be prepared from inorganic and organic acids. Salts derived from inorganic acids include hydrochloric acid, hydrobromic acid, sulfuric acid, nitric acid, phosphoric acid, and the like. Salts derived from organic acids include acetic acid, propionic acid, glycolic acid, pyruvic acid, oxalic acid, malic acid, malonic acid, succinic acid, maleic acid, fumaric acid, tartaric acid, citric acid, benzoic acid, cinnamic acid, mandelic acid, methanesulfonic acid, ethanesulfonic acid, p-toluene-sulfonic acid, salicylic acid, and the like. Likewise, pharmaceutically acceptable base addition salts can be prepared from inorganic and organic bases. Salts derived from inorganic bases include, by way of example only, sodium, potassium, lithium, ammonium, calcium and magnesium salts. Salts derived from organic bases include, but are not limited to, salts of primary, secondary and tertiary amines, such as alkyl amines (i.e., NH₂(alkyl)), dialkyl amines (i.e., HN(alkyl)₂), trialkyl amines (i.e., N(alkyl)₃), substituted alkyl amines (i.e., NH₂(substituted alkyl)), di(substituted alkyl) amines (i.e., HN(substituted alkyl)₂), tri(substituted alkyl) amines (i.e., N(substituted alkyl)₃), alkenyl amines (i.e., NH₂(alkenyl)), dialkenyl amines (i.e., HN(alkenyl)₂), trialkenyl amines (i.e., N(alkenyl)₃), substituted alkenyl amines (i.e., NH₂(substituted alkenyl)), di(substituted alkenyl) amines (i.e., HN(substituted alkenyl)₂), tri(substituted alkenyl) amines (i.e., N(substituted alkenyl)₃, mono-, di- or tri-cycloalkyl amines (i.e., NH₂(cycloalkyl), HN(cycloalkyl)₂, N(cycloalkyl)₃), mono-, di- or tri-arylamines (i.e., NH₂(aryl), HN(aryl)₂, N(aryl)₃), or mixed amines, etc. Specific examples of suitable amines include, by way of example only, isopropylamine, trimethyl amine, diethyl amine, tri(iso-propyl) amine, tri(n-propyl) amine, ethanolamine, 2-dimethylaminoethanol, piperazine, piperidine, morpholine, N-ethylpiperidine, and the like. Similarly, methods of preparing pharmaceutically acceptable salts from an underlying compound (upon disclosure) are known to one of skill in the art and are disclosed in for example, Berge, at al. Journal of Pharmaceutical Science, January 1977 vol. 66, No. 1, and other sources.

As used herein, “pharmaceutically acceptable carrier” includes excipients or agents such as solvents, diluents, dispersion media, coatings, antibacterial and antifungal agents, isotonic and absorption delaying agents and the like that are not deleterious to the disclosed compound or use thereof. The use of such carriers and agents to prepare compositions of pharmaceutically active substances is well known in the art (see, e.g., Remington's Pharmaceutical Sciences, Mace Publishing Co., Philadelphia, Pa. 17th Ed. (1985); and Modern Pharmaceutics, Marcel Dekker, Inc. 3rd Ed. (G. S. Banker & C. T. Rhodes, Eds.).

The terms “therapeutically effective amount” and “effective amount” are used interchangeably and refer to an amount of a compound that is sufficient to effect treatment as defined below, when administered to a patient (e.g., a human) in need of such treatment in one or more doses. The therapeutically effective amount will vary depending upon the patient, the disease being treated, the weight and/or age of the patient, the severity of the disease, or the manner of administration as determined by a qualified prescriber or care giver.

The term “treatment” or “treating” means administering a compound or pharmaceutical composition as described herein for the purpose of: (i) delaying the onset of a disease, that is, causing the clinical symptoms of the disease not to develop or delaying the development thereof; (ii) inhibiting the disease, that is, arresting the development of clinical symptoms; and/or (iii) relieving the disease, that is, causing the regression of clinical symptoms or the severity thereof.

### Liver Diseases

Liver diseases are acute or chronic damages to the liver based in the duration of the disease. The liver damage may be caused by infection, injury, exposure to drugs or toxic compounds such as alcohol or impurities in foods, an abnormal build-up of normal substances in the blood, an autoimmune process, a genetic defect (such as haemochromatosis), or other unknown causes. Exemplary liver diseases include, but are not limited to, cirrhosis, liver fibrosis, non-alcoholic fatty liver disease (NAFLD), non-alcoholic steatohepatitis (NASH), alcoholic steatohepatitis (ASH), hepatic ischemia reperfusion injury, primary biliary cirrhosis (PBC), and hepatitis, including both viral and alcoholic hepatitis.

Non-alcoholic fatty liver disease (NAFLD) is the build up of extra fat in liver cells that is not caused by alcohol. NAFLD is characterized by the accumulation of fat in hepatocytes and is often associated with some aspects of metabolic syndrome (e.g. type 2 diabetes mellitus, insulin resistance, hyperlipidemia, hypertension). The frequency of this disease has become increasingly common due to consumption of carbohydrate-rich and high fat diets. A subset of NAFLD patients develop nonalcoholic steatohepatitis (NASH).

NASH, a subtype of fatty liver disease, is the more severe form of NAFLD. It is characterized by macrovesicular steatosis, balloon degeneration of hepatocytes, and/or inflammation ultimately leading to hepatic scarring (i.e. fibrosis). Patients diagnosed with NASH progress to advanced stage liver fibrosis and eventually cirrhosis. The current treatment for cirrhotic NASH patients with end-stage disease is liver transplant.

Another common liver disease is primary sclerosing cholangitis (PSC). It is a chronic or long-term liver disease that slowly damages the bile ducts inside and outside the liver. In patients with PSC, bile accumulates in the liver due to blocked bile ducts, where it gradually damages liver cells and causes cirrhosis, or scarring of the liver. Currently, there is no effective treatment to cure PSC. Many patients having PSC ultimately need a liver transplant due to liver failure, typically about 10 years after being diagnosed with the disease. PSC may also lead to bile duct cancer.

Liver fibrosis is the excessive accumulation of extracellular matrix proteins, including collagen, which occurs in most types of chronic liver diseases. Advanced liver fibrosis results in cirrhosis, liver failure, and portal hypertension and often requires liver transplantation.

### Methods

Disclosed herein is a method of treating and/or preventing liver disease in a patient in need thereof, comprising administering to the patient a therapeutically effective amount of an ASK1 inhibitor in combination with a therapeutically effective amount of an ACC inhibitor. The presence of active liver disease can be detected by the existence of elevated enzyme levels in the blood. Specifically, blood levels of alanine aminotransferase (ALT) and aspartate aminotransferase (AST) above clinically accepted normal ranges are known to be indicative of on-going liver damage. Routine monitoring of liver disease patients for blood levels of ALT and AST is used clinically to measure progress of the liver disease while on medical treatment. Reduction of elevated ALT and AST to within the accepted normal range is taken as clinical evidence reflecting a reduction in the severity of the patient's on-going liver damage.

In certain embodiments, the liver disease is a chronic liver disease. Chronic liver diseases involve the progressive destruction and regeneration of the liver parenchyma, leading to fibrosis and cirrhosis. In general, chronic liver diseases can be caused by viruses (such as hepatitis B, hepatitis C, cytomegalovirus (CMV), or Epstein Barr Virus (EBV)), toxic agents or drugs (such as alcohol, methotrexate, or nitrofurantoin), a metabolic disease (such as non-alcoholic fatty liver disease (NAFLD), non-alcoholic steatohepatitis (NASH), haemochromatosis, or Wilson's Disease), an autoimmune disease (such as Autoimmune Chronic Hepatitis, Primary Biliary Cholangitis (formerly known as Primary Biliary Cirrhosis), or Primary Sclerosing Cholangitis), or other causes (such as right heart failure).

In one embodiment, provided herein is a method for reducing the level of cirrhosis. In one embodiment, cirrhosis is characterized pathologically by loss of the normal microscopic lobular architecture, with fibrosis and nodular regeneration. Methods for measuring the extent of cirrhosis are well known in the art. In one embodiment, the level of cirrhosis is reduced by about 5% to about 100%. In one embodiment, the level of cirrhosis is reduced by at least about 5%, at least about 10%, at least about 15%, at least about 20%, at least about 25%, at least about 30%, at least about 35%, at least about 40%, at least about 45%, at least 50%, at least about 55%, at least about 60%, at least about 65%, at least about 70%, at least about 75%, at least about 80%, at least about 85%, at least about 90%, at least about 95% in the subject.

In certain embodiments, the liver disease is a metabolic liver disease. In one embodiment, the liver disease is non-alcoholic fatty liver disease (NAFLD). NAFLD is associated with insulin resistance and metabolic syndrome (obesity, combined hyperlipidemia, diabetes mellitus (type II) and high blood pressure). NAFLD is considered to cover a spectrum of disease activity, and begins as fatty accumulation in the liver (hepatic steatosis).

It has been shown that both obesity and insulin resistance probably play a strong role in the disease process of NAFLD. In addition to a poor diet, NAFLD has several other known causes. For example, NAFLD can be caused by certain medications, such as amiodarone, antiviral drugs (e.g., nucleoside analogues), aspirin (rarely as part of Reye's syndrome in children), corticosteroids, methotrexate, tamoxifen, or tetracycline. NAFLD has also been linked to the consumption of soft drinks through the presence of high fructose corn syrup which may cause increased deposition of fat in the abdomen, although the consumption of sucrose shows a similar effect (likely due to its breakdown into fructose). Genetics has also been known to play a role, as two genetic mutations for this susceptibility have been identified.

If left untreated, NAFLD can develop into non-alcoholic steatohepatitis (NASH). NASH is regarded as a major cause of cirrhosis of the liver. Accordingly, provided herein is a method of treating and/or preventing nonalcoholic steatohepatitis (NASH) in a patient in need thereof, comprising administering to the patient a therapeutically effective amount of an ASK1 inhibitor in combination with a therapeutically effective amount of an ACC inhibitor.

Also provided herein is a method of treating and/or preventing liver fibrosis in a patient in need thereof, comprising administering to the patient a therapeutically effective amount of an ASK1 inhibitor in combination with a therapeutically effective amount of an ACC inhibitor. Liver fibrosis is the excessive accumulation of extracellular matrix proteins including collagen that occurs in most types of chronic liver diseases. In certain embodiments, advanced liver fibrosis results in cirrhosis and liver failure. Methods for measuring liver histologies, such as changes in the extent of fibrosis, lobular hepatitis, and periportal bridging necrosis, are well known in the art.

In one embodiment, the level of liver fibrosis, which is the formation of fibrous tissue, fibroid or fibrous degeneration, is reduced by more that about 90%. In one embodiment, the level of fibrosis, which is the formation of fibrous tissue, fibroid or fibrous degeneration, is reduced by at least about 90%, at least about 80%, at least about 70%, at least about 60%, at least about 50%, at least about 40%, at least about 30%, at least about 20%, at least about 10%, at least about 5% or at least about 2%.

Some embodiments described herein are directed to a method of treating liver disease comprising administering a therapeutically effective amount of a form of Compound I as described herein or a pharmaceutical composition as described herein. Liver disease can be classified into 4 stages: F0 indicates no fibrosis; F1 indicates mild fibrosis; F2 indicates moderate fibrosis; F3 indicates severe fibrosis; and F4 indicates cirrhosis. In one embodiment, the level of liver fibrosis as measured by fibrosis stage is reduced from baseline. In one embodiment, the liver fibrosis stage improvement is greater than 1 from baseline or greater than 2 from baseline after daily treatment over a period of time. In another embodiment, liver fibrosis stage is improved from F4 to F3, from F3 to F2, or from F2 to F1 by a method comprising administering to the patient a therapeutically effective amount of an ASK1 inhibitor in combination with a therapeutically effective amount of an ACC inhibitor. In another embodiment, provided is a method for treating liver fibrosis in a patient in need thereof, wherein the liver fibrosis stage of the patient is F3, comprising administering to the patient a therapeutically effective amount of an ASK1 inhibitor in combination with a therapeutically effective amount of an ACC inhibitor. In another embodiment, provided is a method for treating liver fibrosis in a patient in need thereof, wherein the liver fibrosis stage of the patient is F4, comprising administering to the patient a therapeutically effective amount of an ASK1 inhibitor in combination with a therapeutically effective amount of an ACC inhibitor. In one embodiment, the liver fibrosis stage improvement from baseline is greater than 1 after 24 weeks of daily administration of a therapeutically effective amount of an ASK1 inhibitor in combination with a therapeutically effective amount of an ACC inhibitor. In another embodiment, liver fibrosis stage improvement from baseline is greater than 1 after 48 or 92 weeks of daily administration of an ASK1 inhibitor in combination with a therapeutically effective amount of an ACC inhibitor as described herein. In still another embodiment, liver fibrosis stage improvement from baseline is greater than 1 after 48 or 92 weeks of daily administration of an ASK1 inhibitor in combination with a therapeutically effective amount of an ACC inhibitor as described herein.

In one embodiment, the compounds provided herein reduce the level of fibrogenesis in the liver. Liver fibrogenesis is the process leading to the deposition of an excess of extracellular matrix components in the liver known as fibrosis. It is observed in a number of conditions such as chronic viral hepatitis B and C, alcoholic liver disease, drug-induced liver disease, hemochromatosis, auto-immune hepatitis, Wilson disease, Primary Biliary Cholangitis (formerly known as Primary Biliary Cirrhosis), sclerosing cholangitis, liver schistosomiasis and others. In one embodiment, the level of fibrogenesis is reduced by more that about 90%. In one embodiment, the level of fibrogenesis is reduced by at least about 90%, at least about 80%, at least about 70%, at least about 60%, at least about 50%, at least 40%, at least about 30%, at least about 20%, at least about 10%, at least about 5% or at least 2%.

In still other embodiments, provided herein is a method of treating and/or preventing primary sclerosing cholangitis (PSC) in a patient in need thereof, comprising administering to the patient a therapeutically effective amount of an ASK1 inhibitor in combination with a therapeutically effective amount of an ACC inhibitor.

It has been observed that patients having NASH are on average about 2.8 years older than healthy patients in epigenetic testing. Thus, in one embodiment, compounds useful for the treatment of NASH would be useful for slowing, improving or reversing epigenetic age or effects of aging due to NASH. In another embodiment, combination therapies for the treatment of NASH such as, for example, the combination of an ASK1 inhibitor compound with an ACC inhibitor compound as disclosed herein would be useful for improvement or reversal of aging effects due to NASH.

In one embodiment, the ASK1 inhibitor and the ACC inhibitor may be administered together in a combination formulation or in separate pharmaceutical compositions, where each inhibitor may be formulated in any suitable dosage form. In certain embodiments, the methods provided herein comprise administering separately a pharmaceutical composition comprising an ASK1 inhibitor and a pharmaceutically acceptable carrier or excipient and a pharmaceutical composition comprising an ACC inhibitor and a pharmaceutically acceptable carrier or excipient. Combination formulations according to the present disclosure comprise an ASK1 inhibitor and an ACC inhibitor together with one or more pharmaceutically acceptable carriers or excipients and optionally other therapeutic agents. Combination formulations containing the active ingredient may be in any form suitable for the intended method of administration.

Fibrosis improvement may be measured by magnetic resonance elastography (MRE). MRE can be used to discriminate stiffness for a fibrosis improvement of ≥1-stage. In one embodiment, the disclosure provides a method of administering to a human a compound selected from a compound of Formula (I), a compound of Formula (II), a compound of Formula (III), a compound of Formula (IV) to a patient diagnosed with NASH, and measuring fibrosis improvement by MRE. In one embodiment, the disclosure provides a method of administering to a human a compound of Formula (I) or a compound of Formula (II) in combination, or concurrently with, a compound of Formula (III) or a compound of Formula (IV), to a patient diagnosed with NASH, and measuring fibrosis improvement by MRE. In another embodiment, the disclosure provides a method diagnosing a human with NASH by MRE and administering a compound of Formula (I), a compound of Formula (II), a compound of Formula (III), a compound of Formula (IV) or a combination thereof to treat NASH. The AUROC of MRE-stiffness to predict fibrosis improvement is 0.62 (95% CI 0.45-0.78) and the optimal threshold is a ≥0% relative reduction.

Histologic improvements can also be measured by proton density fat fraction (PDFF). PDFF can be used to predict steatosis improvement of ≥1-grade. PDFF can also be used to predicted NAS responses (≥2 point reductions). In one embodiment the disclosure provides a method of administering a compound selected from a compound of Formula (I), a compound of Formula (II), a compound of Formula (III), a compound of Formula (IV) or combinations thereof to provide a ≥1-grade steatosis improvement. In another embodiment, the disclosure provides a method of administering a compound selected from a compound of Formula (I), a compound of Formula (II), a compound of Formula (III), a compound of Formula (IV) or combinations thereof to provide a ≥2 point reduction in NAS score. The AUROC of PDFF to predict NAS response is 0.70 (95% CI 0.51-0.89) and the optimal threshold is a ≥25% relative reduction. For steatosis improvement the AUROC of PDFF is 0.70 (95% CI 57-83) and the optimum threshold is a ≥0% relative reduction.

### ASK1 Inhibitors

In certain embodiments of the methods and pharmaceutical compositions disclosed herein, the ASK1 inhibitor is a compound having the structure of Formula (I):

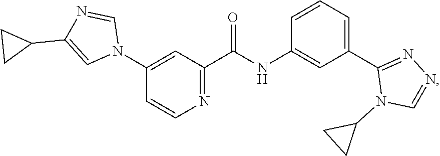 [SMILES: O=C(Nc1cccc(-c2nncn2C2CC2)c1)c1cc(-n2cnc(C3CC3)c2)ccn1]

or a pharmaceutically acceptable salt thereof.

In certain embodiments of the methods and pharmaceutical compositions disclosed herein, the ASK1 inhibitor is a compound having the structure of Formula (II):

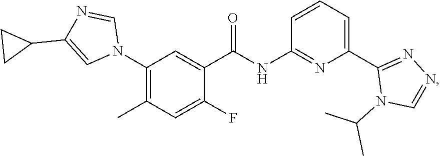 [SMILES: Cc1cc(F)c(C(=O)Nc2cccc(-c3nncn3C(C)C)n2)cc1-n1cnc(C2CC2)c1]

or a pharmaceutically acceptable salt thereof.

The compounds of Formula (I) and Formula (II) may be synthesized and characterized using methods known to those of skill in the art, such as those described in U.S. Pat. No. 8,742,126 and U.S. Patent Application Publication Nos. 2011/0009410 and 2013/0197037. In one embodiment, the ASK1 inhibitor is the compound of Formula (I) or a pharmaceutically acceptable salt thereof. In one embodiment, the ASK1 inhibitor is the compound of Formula (II) or a pharmaceutically acceptable salt thereof.

### ACC Inhibitors

In certain embodiments of the methods and pharmaceutical compositions disclosed herein, the ACC inhibitor is a compound having the structure of Formula (III):

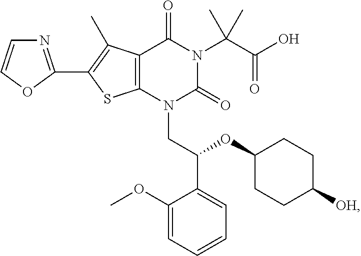 [SMILES: COc1ccccc1[C@H](Cn1c(=O)n(C(C)(C)C(=O)O)c(=O)c2c(C)c(-c3ncco3)sc21)O[C@H]1CC[C@@H](O)CC1]

or a pharmaceutically acceptable salt thereof.

In certain embodiments of the methods and pharmaceutical compositions disclosed herein, the ACC inhibitor is a compound having the structure of Formula (IV):

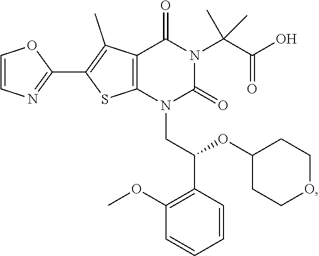 [SMILES: COc1ccccc1[C@H](Cn1c(=O)n(C(C)(C)C(=O)O)c(=O)c2c(C)c(-c3ncco3)sc21)OC1CCOCC1]

or a pharmaceutically acceptable salt thereof.

The compounds of Formula (III) and Formula (IV) may be synthesized and characterized using methods known to those of skill in the art, such as those described in International Application Publication No. WO/2013071169.

### Dosing and Administration

While it is possible for an active ingredient to be administered alone, they may be administered as pharmaceutical formulations or pharmaceutical compositions as described below. The formulations, both for veterinary and for human use, of the disclosure comprise at least one of the active ingredients, together with one or more acceptable carriers therefor and optionally other therapeutic ingredients. The carrier(s) must be “acceptable” in the sense of being compatible with the other ingredients of the formulation and physiologically innocuous to the recipient thereof.

Each of the active ingredients can be formulated with conventional carriers and excipients, which will be selected in accord with ordinary practice. Tablets can contain excipients, glidants, fillers, binders and the like. Aqueous formulations are prepared in sterile form, and when intended for delivery by other than oral administration generally will be isotonic. All formulations will optionally contain excipients such as those set forth in the Handbook of Pharmaceutical Excipients (1986). Excipients include ascorbic acid and other antioxidants, chelating agents such as EDTA, carbohydrates such as dextrin, hydroxyalkylcellulose, hydroxyalkylmethylcellulose, stearic acid and the like. The pH of the formulations ranges from about 3 to about 11, but is ordinarily about 7 to 10.

The therapeutically effective amount of active ingredient can be readily determined by a skilled clinician using conventional dose escalation studies. Typically, the active ingredient (i.e., a compound as described herein) will be administered in a dose from 0.01 milligrams to 2 grams. In one embodiment, the dosage will be from about 10 milligrams to 450 milligrams. In another embodiment, the dosage will be from about 25 to about 250 milligrams. In another embodiment, the dosage will be about 50 or 100 milligrams. In one embodiment, the dosage will be about 100 milligrams. It is contemplated that the active ingredient may be administered once, twice or three times a day. Also, the active ingredient may be administered once or twice a week, once every two weeks, once every three weeks, once every four weeks, once every five weeks, or once every six weeks. In one embodiment, the dose of the ASK1 inhibitor is from 6 to 25 milligrams and the dose of the ACC inhibitor is from 5 to 30 milligrams. In one embodiment, the dose of the ASK1 inhibitor is about 6 milligrams and the dose of the ACC inhibitor is about 10 milligrams. In one embodiment, the dose of the ASK1 inhibitor is about 6 milligrams and the dose of the ACC inhibitor is about 20 milligrams. In one embodiment, the dose of the ASK1 inhibitor is 18 milligrams and the dose of the ACC inhibitor is 20 milligrams. In certain embodiments, the dose is the total daily dose.

The pharmaceutical composition for the active ingredient can include those suitable for the foregoing administration routes. The formulations can conveniently be presented in unit dosage form and may be prepared by any of the methods well known in the art of pharmacy. Techniques and formulations generally are found in Remington's Pharmaceutical Sciences (Mack Publishing Co., Easton, Pa.). Such methods include the step of bringing into association the active ingredient with the carrier which constitutes one or more accessory ingredients. In general the formulations are prepared by uniformly and intimately bringing into association the active ingredient with liquid carriers or finely divided solid carriers or both, and then, if necessary, shaping the product.

Formulations suitable for oral administration can be presented as discrete units such as capsules, cachets or tablets each containing a predetermined amount of the active ingredient; as a powder or granules; as a solution or a suspension in an aqueous or non-aqueous liquid; or as an oil-in-water liquid emulsion or a water-in-oil liquid emulsion. The active ingredient may also be administered as a bolus, electuary or paste. In certain embodiments, the active ingredient may be administered as a subcutaneous injection.

A tablet can be made by compression or molding, optionally with one or more accessory ingredients. Compressed tablets can be prepared by compressing in a suitable machine the active ingredient in a free-flowing form such as a powder or granules, optionally mixed with a binder, lubricant, inert diluent, preservative, or surface active agent. Molded tablets may be made by molding in a suitable machine a mixture of the powdered active ingredient moistened with an inert liquid diluent. The tablets may optionally be coated or scored and optionally are formulated so as to provide slow or controlled release of the active ingredient therefrom.

The active ingredient can be administered by any route appropriate to the condition. Suitable routes include oral, rectal, nasal, topical (including buccal and sublingual), vaginal and parenteral (including subcutaneous, intramuscular, intravenous, intradermal, intrathecal and epidural), and the like. It will be appreciated that the preferred route may vary with for example the condition of the recipient. In certain embodiments, the active ingredients are orally bioavailable and can therefore be dosed orally. In one embodiment, the patient is human.

When used in combination in the methods disclosed herein, the ASK1 inhibitor and the ACC inhibitor can be administered together in a single pharmaceutical composition or separately (either concurrently or sequentially) in more than one pharmaceutical composition. In certain embodiments, the ASK1 inhibitor and the ACC inhibitor are administered together. In other embodiments, the ASK1 inhibitor and the ACC inhibitor are administered separately. In some aspects, the ASK1 inhibitor is administered prior to the ACC inhibitor. In some aspects, the ACC inhibitor is administered prior to the ASK1 inhibitor. When administered separately, the ASK1 inhibitor and the ACC inhibitor can be administered to the patient by the same or different routes of delivery.

### Pharmaceutical Compositions

The pharmaceutical compositions of the disclosure comprise an effective amount of an ASK1 inhibitor selected from the group consisting of a compound of Formula (I) and a compound of Formula (II), and an effective amount of an ACC inhibitor selected from the group consisting of a compound of Formula (III) and a compound of Formula (IV).

When used for oral use for example, tablets, troches, lozenges, aqueous or oil suspensions, dispersible powders or granules, emulsions, hard or soft capsules, syrups or elixirs may be prepared. Compositions intended for oral use may be prepared according to any method known to the art for the manufacture of pharmaceutical compositions and such compositions may contain one or more agents including sweetening agents, flavoring agents, coloring agents and preserving agents, in order to provide a palatable preparation. Tablets containing the active ingredient in admixture with non-toxic pharmaceutically acceptable excipient which are suitable for manufacture of tablets are acceptable. These excipients may be, for example, inert diluents, such as, for example, calcium or sodium carbonate, lactose, lactose monohydrate, croscarmellose sodium, povidone, calcium or sodium phosphate; granulating and disintegrating agents, such as, for example, maize starch, or alginic acid; binding agents, such as, for example, cellulose, microcrystalline cellulose, starch, gelatin or acacia; and lubricating agents, such as, for example, magnesium stearate, stearic acid or talc. Tablets may be uncoated or may be coated by known techniques including microencapsulation to delay disintegration and adsorption in the gastrointestinal tract and thereby provide a sustained action over a longer period. For example, a time delay material such as, for example, glyceryl monostearate or glyceryl distearate alone or with a wax may be employed.

Formulations for oral use may be also presented as hard gelatin capsules where the active ingredient is mixed with an inert solid diluent, for example calcium phosphate or kaolin, or as soft gelatin capsules wherein the active ingredient is mixed with water or an oil medium, such as, for example, peanut oil, liquid paraffin or olive oil.

Aqueous suspensions of the disclosure contain the active materials in admixture with excipients suitable for the manufacture of aqueous suspensions. Such excipients include a suspending agent, such as, for example, sodium carboxymethylcellulose, methylcellulose, hydroxypropyl methylcelluose, sodium alginate, polyvinylpyrrolidone, gum tragacanth and gum acacia, and dispersing or wetting agents such as, for example, a naturally occurring phosphatide (e.g., lecithin), a condensation product of an alkylene oxide with a fatty acid (e.g., polyoxyethylene stearate), a condensation product of ethylene oxide with a long chain aliphatic alcohol (e.g., heptadecaethyleneoxycetanol), a condensation product of ethylene oxide with a partial ester derived from a fatty acid and a hexitol anhydride (e.g., polyoxyethylene sorbitan monooleate). The aqueous suspension may also contain one or more preservatives such as, for example, ethyl or n-propyl p-hydroxy-benzoate, one or more coloring agents, one or more flavoring agents and one or more sweetening agents, such as, for example, sucrose or saccharin.

Oil suspensions may be formulated by suspending the active ingredient in a vegetable oil, such as, for example, arachis oil, olive oil, sesame oil or coconut oil, or in a mineral oil such as, for example, liquid paraffin. The oral suspensions may contain a thickening agent, such as, for example, beeswax, hard paraffin or cetyl alcohol. Sweetening agents, such as, for example, those set forth above, and flavoring agents may be added to provide a palatable oral preparation. These compositions may be preserved by the addition of an antioxidant such as, for example, ascorbic acid.

Dispersible powders and granules of the disclosure suitable for preparation of an aqueous suspension by the addition of water provide the active ingredient in admixture with a dispersing or wetting agent, a suspending agent, and one or more preservatives. Suitable dispersing or wetting agents and suspending agents are exemplified by those disclosed above. Additional excipients, for example sweetening, flavoring and coloring agents, may also be present.

The pharmaceutical compositions of the disclosure may also be in the form of oil-in-water emulsions. The oily phase may be a vegetable oil, such as, for example, olive oil or arachis oil, a mineral oil, such as, for example, liquid paraffin, or a mixture of these. Suitable emulsifying agents include naturally-occurring gums, such as, for example, gum acacia and gum tragacanth, naturally occurring phosphatides, such as, for example, soybean lecithin, esters or partial esters derived from fatty acids and hexitol anhydrides, such as, for example, sorbitan monooleate, and condensation products of these partial esters with ethylene oxide, such as, for example, polyoxyethylene sorbitan monooleate. The emulsion may also contain sweetening and flavoring agents. Syrups and elixirs may be formulated with sweetening agents, such as, for example, glycerol, sorbitol or sucrose. Such formulations may also contain a demulcent, a preservative, a flavoring or a coloring agent.

The pharmaceutical compositions of the disclosure may be in the form of a sterile injectable preparation, such as, for example, a sterile injectable aqueous or oleaginous suspension. This suspension may be formulated according to the known art using those suitable dispersing or wetting agents and suspending agents which have been mentioned above. The sterile injectable preparation may also be a sterile injectable solution or suspension in a non-toxic parenterally acceptable diluent or solvent, such as, for example, a solution in 1,3-butane-diol or prepared as a lyophilized powder. Among the acceptable vehicles and solvents that may be employed are water, Ringer's solution and isotonic sodium chloride solution. In addition, sterile fixed oils may conventionally be employed as a solvent or suspending medium. For this purpose any bland fixed oil may be employed including synthetic mono- or diglycerides. In addition, fatty acids such as, for example, oleic acid may likewise be used in the preparation of injectables.

The amount of active ingredient that may be combined with the carrier material to produce a single dosage form will vary depending upon the host treated and the particular mode of administration, such as oral administration or subcutaneous injection. For example, a time-release formulation intended for oral administration to humans may contain approximately 1 to 1000 mg of active material compounded with an appropriate and convenient amount of carrier material which may vary from about 5 to about 95% of the total compositions (weight:weight). The pharmaceutical composition can be prepared to provide easily measurable amounts for administration. For example, an aqueous solution intended for intravenous infusion may contain from about 3 to 500 μg of the active ingredient per milliliter of solution in order that infusion of a suitable volume at a rate of about 30 mL/hr can occur. When formulated for subcutaneous administration, the formulation is typically administered about twice a month over a period of from about two to about four months.

Formulations suitable for parenteral administration include aqueous and non-aqueous sterile injection solutions which may contain anti-oxidants, buffers, bacteriostats and solutes which render the formulation isotonic with the blood of the intended recipient; and aqueous and non-aqueous sterile suspensions which may include suspending agents and thickening agents.

The formulations can be presented in unit-dose or multi-dose containers, for example sealed ampoules and vials, and may be stored in a freeze-dried (lyophilized) condition requiring only the addition of the sterile liquid carrier, for example water for injection, immediately prior to use. Extemporaneous injection solutions and suspensions are prepared from sterile powders, granules and tablets of the kind previously described. Preferred unit dosage formulations are those containing a daily dose or unit daily sub-dose, as herein above recited, or an appropriate fraction thereof, of the active ingredient.

### EXAMPLES

### Example 1. Efficacy in a Mouse Model of NASH

The following study was conducted to evaluate the efficacy of the combination of an ASK1 inhibitor and an ACC inhibitor in a mouse model of non-alcoholic steatohepatitis (NASH), relative to the efficacy of the individual agents alone in the model. NASH was induced in male C57BL/6 mice by chronic administration of a “fast food” diet (FFD) high in saturated fats, cholesterol and sugars for a total of 6 months, whereas lean control animals were maintained on a normal chow diet. A NASH phenotype was established in FFD mice compared to control mice after 6 months, and was characterized by macrovesicular steatosis, elevated ALT and AST, and increased levels of transcripts associated with hepatic stellate cell activation. See Charlton M, et al. Fast food diet mouse: novel small animal model of NASH with ballooning, progressive fibrosis, and high physiological fidelity to the human condition. American Journal of Physiology. Gastrointestinal and Liver Physiology 2011; 301 (5):G825-34.

After 5 months, FFD mice were subsequently treated with placebo (vehicle), an ASK1 inhibitor (Formula (I)), an ACC inhibitor (Formula (III)), or with the combination of Formula (I) and Formula (III) for 1 month. Control mice remained on a normal chow diet for the entire 6 month study period. Endpoint analyses included morphometric quantification of liver steatosis (% of steatotic area), liver triglycerides, liver cholesterol, ALT, and measurement of the pro-fibrotic transcripts Timp1 and CollA1.

### Methods

### Animals

Male C57CL/6 mice (aged 12 weeks at study inception) were used in this study. All procedures used to study the animals were in the compliance with the U.S. Department of Agriculture's Animal Welfare Act (9 CFR Parts 1, 2, and 3); the Guide for the Care and Use of Laboratory Animals (Institute for Laboratory Animal Research, The National Academies Press, Washington, D.C.); and the National Institutes of Health, Office of Laboratory Animal Welfare.

### In-Life Experimental Protocol for the FFD Mouse Model

The experimental design is shown in Table 1. Study animals were administered either a standard chow diet (Harlan Teklad Global Diets 2014, TD2014) or a commercially available high fat, high cholesterol diet (Research Diets Inc, DB12079B) hereafter referred to as the NASH diet. Animals were administered fructose/glucose in drinking water formulated as follows: 23.1 g fructose (Sigma, F2543) and 17.2 g of glucose (Sigma, 49158) was mixed into 1000 mL of drinking water.

Treatment with the compound of Formula (I) or the compound of Formula (III) alone, or the combination of the compounds of Formula (I) and Formula (III) were administered for the final month of the study (month 5-month 6). The compound of Formula (I) was administered as a 0.15% admixture in the FFD diet, the compound of Formula (III) was formulated in 0.5% sodium carboxymethylcellulose (medium viscosity), 1% ethanol, 98.5% 50 mM Tris buffer, pH8 in reverse osmosis water. The compound of Formula (III) was formulated at either 0.1 or 0.2 mg/mL and given in the dose provided on the table.

Five days before PO dosing started, groups 2 and 5 were placed on DB12079B containing 0.15% of the compound of Formula (I) milled into the diet for 35 days. The week-long lead-in dosing of the compound of Formula (I) was designed to overcome food aversion during the first week of introducing the compound of Formula (I). Starting seven days before PO dosing, animals in groups 1-7 were sham dosed with vehicle BID. The sham dosing was designed to acclimate animals to oral gavage dose administration. Starting at Day 1 of the study, animals in all dose groups were dosed three times daily; twice sequentially in the AM (7:00+/−1 hour), and once in the evening (19:00+/−1 hr), with the same volume of formulation containing no compound (group 1, vehicle) or the appropriate compounds as outlined below (Table 1) for 28 days (until dosing Day 29). Each group was split into two and half were sacrificed 2 hours post dose, and half were sacrificed 8 hours post dose on Day 29.

TABLE 1
Experimental Design and Dose Groups

| | | | Dose | | Number | Dosing | Dosing | |
|---|---|---|---|---|---|---|---|---|
| | | Dose | Vol | Concentration | of | Frequency | Duration | |
| Group | Test Article | (mg/kg) | (mL/kg) | (mg/mL) | Animals | (x/day) | (days) | Route |
| 1 | Vehicle | 0 | 5 | 0 | 15 | TID | 29 | PO |
| 2 | Vehicle | 0 | 5 | 0 | 15 | TID | 29 | PO |
| | Formula (I) | Ad libitum | NA | 0.15 | | Ad libitum | 32 | Diet |
| 3 | Vehicle | 0 | 5 | 0 | 15 | QD | 29 | PO |
| | Formula (III) | 0.5 | 5 | 0.1 | | BID | 29 | PO |
| 4 | Vehicle | 0 | 5 | 0 | 15 | QD | 29 | PO |
| | Formula (III) | 1 | 5 | 0.2 | | BID | 29 | PO |
| 5 | Vehicle | 0 | 5 | 0 | 16 | QD | 29 | PO |
| | Formula (III) | 0.5 | 5 | 0.1 | | BID | 29 | PO |
| | Formula (I) | Ad libitum | NA | 0.15 | | Ad libitum | 32 | Diet |
| 6 | Vehicle (age- | 0 | 5 | 0 | 10 | TID | 29 | PO |
| | matched lean) | | | | | | | |
| 7 | Vehicle | 0 | 5 | 0 | 10 | TID | 29 | PO |
| | (young lean) | | | | | | | |

### Measurement of Hepatic Steatosis

Whole slide-scan images of Hematoxylin & Eosin (H&E) stained slides were captured using an AperioAT2 scanner at 40× magnification (Leica Biosystems, Buffalo Grove, Ill.). Digital slide images were checked for scanning quality, annotated and exported to appropriate network folders within Leica Digital Image Hub (Leica Biosystems) archive. Quantification of steatosis was performed on the whole slide-scan images using Visiopharm image analysis software (Visopharm, Hoersholm, Denmark). Lipid vacuoles within the liver parenchyma were round areas of low optical density (white). The number and size of these areas were enumerated and the total steatotic area was expressed as a percentage of total liver tissue cross-sectional area. Intrahepatic vessels (such as branches of the portal vein and central vein) were excluded from this analysis based upon size and shape. The masks of the automated analysis were individually reviewed to confirm accuracy of the results. Hepatic Steatosis was determined on animals sacrificed at 8 hourse post dose (half of each group). All other analyses were performed on all animals.

Quantification of Triglycerides and Cholesterol from Murine Liver

Tissue Extraction: Mouse liver tissue samples (25±10 mg, accurately weighed in frozen state) were homogenized and extracted with a water immiscible organic solvent mixture that extracts the triacylglyceride fraction as well as the free and esterified cholesterol fractions into the organic phase. After centrifugation, an aliquot of the organic upper layer, containing the triacylglycerides, cholesterol and cholesterol esters was diluted either 10-fold or 25-fold with ethanol. Two separate aliquots of this dilution were taken. One aliquot was analyzed for triacylglycerides, the second aliquot was used for the total cholesterol determination.

Triacylglyceride Determination: For the triacylglyceride determination, one aliquot of the 25-fold dilution (or no dilution in the case of samples which have low triacylglyceride content) was evaporated under a stream of nitrogen. The dried extract was reconstituted stepwise with a 0.1% sodium dodecyl sulfate in PBS solution under ultrasonication followed by mixing with the Triacylglyceride Determination Reagent (Infinity™ Triglycerides Liquid Stable Reagent, Thermo Scientific, Product Data Sheet, Infinity™, Triglycerides Liquid Stable Reagent).

This reagent solution contained several enzymes, cofactors and the chromogenic reagent 4-aminoantipyrine. The determination of triacylglycerides (TAG) with this reagent was based on the method of Wako, Product Data Sheet, Triacylglyceride—G Code No. 997-69801, Wako Pure Chemical Industries Ltd., Dallas, Tex., and the modifications by McGowan et al, (McGowan, M W, et al., Clin. Chem 1983:29:538) and Fossati et al (Fosseti, P. Prenciple L. Clin Chem. 1892:28:2077-80) as follows:
1. Triglycerides are enzymatically hydrolyzed by lipase to free fatty acids and glycerol.
2. The glycerol is phosphorylated by adenosine triphosphate (ATP) with glycerol kinase (GK) to produce glycerol-3-phosphate and adenosine diphosphate.
3. Glycerol-3-phosphate is oxidized by dihydroxyacetone phosphate (DAP) by glycerol phosphate oxidase producing hydrogen peroxide (H₂O₂).
4. In a Trinder⁵-type colour reaction catalyzed by peroxidase, the H₂O₂ reacts with 4-aminoantipyrine (4-AAP) and 3,5-dichloro-2-hydroxybenzene sulfonate (DHBS) to produce a red colored dye. The absorbance of this dye is proportional to the concentration of triglycerides present in the sample.

After incubation with the Triacylglyceride Determination Reagent for 30 min at 37° C., samples were transferred into a microtiter plate, and the absorbance is measured at 540 nm in a microplate reader (SpectraMax M2, Molecular Devices). Quantitation was performed using a linear least squares regression analysis generated from fortified calibration standards using glyceryl trioleate (triolein) as triacylglyceride reference standard. Calibration standard samples were taken through the same extraction and incubation steps as the tissue samples. Weight corrections and concentration calculations were performed using Microsoft Excel 2013. Final tissue contents were given in μmol Triacylglyceride (TAG)/g Liver Tissue.

Total Cholesterol Determination: For the total cholesterol determination, internal standard solution (cholesterol-d₆) and 1 M ethanolic potassium hydroxide solution were added to an aliquot of the 10-fold sample dilution (see above). The mixture was incubated at 70° C. for one hour in order to hydrolyze the cholesterol esters to free fatty acids and cholesterol. Afterwards, the reaction mixture was acidified with glacial acetic acid and extracted with hexanes. The hexanes layer was removed, evaporated and reconstituted in acetonitrile.

An aliquot of the reconstituted extract was injected onto a Waters Acquity/AB Sciex QTrap 4000 LC-MS/MS system equipped with a C18 reversed phase column. The mass spectrometer was operated in positive mode using atmospheric pressure chemical ionization (APCI).

The peak area of the m/z 369 [M-H₂O]⁺43 161⁺ product ion of cholesterol was measured against the peak area of the cholesterol-D₆ product ion of m/z 375 [M-H₂O]⁺→167⁺. Quantitation was performed using a weighted (1/×) linear least squares regression analysis generated from fortified calibration standards using cholesteryl oleate as reference standard. Calibration standard samples were taken through the same extraction and hydrolysis steps as the tissue samples. Raw data were collected and processed using AB SCIEX software Analyst 1.5.1. Data reduction, weight corrections, correction for cholesteryl oleate to cholesterol hydrolysis and concentration calculations were performed using Microsoft Excel 2013. Final tissue contents were given in mg Total Cholesterol/g Liver Tissue

### ALT

Serum was collected from all mice at terminal necroscopy. Serum ALT was measured by Pyruvate with pyridoxal-5′-phosphate and analyzed on the Cobas Hitachi 6000 Chemistry System, Roche Diagnostics.

### Gene Expression

An approximately 100 mg chunk of frozen left lateral lobe was sent to DC3 for lysing and RNA extraction. NanoString assays were carried out with all reagents and consumables contained in an nCounter master kit (NanoString) according to manufacturer instructions to measure RNA transcripts. Briefly, the color coded reporter probe targeting 110 liver fibrosis related genes and 6 control housekeeping genes (Table 2) were hybridized overnight in a pre-heated 65° C. thermocycler for 16 to 22 hours with 100 ng RNA samples in a reaction that includes a hybridization buffer and a capture probe. Following incubation, samples were placed on a prep station where excess probes were removed and the probe-transcript complexes were immobilized on a streptavidin coated cartridge. Finally, the cartridges were imaged in the nCounter Digital Analyzer (NanoString Technologies, Seattle, Wash.). All transcripts were normalized to the geometric mean of 6 housekeeping genes (B2m, HPRT, Pgk1, RPL13a, Rpn1, and SFRS4) with nSover 3.0 software.

TABLE 2
Nanostring Probes

| | | | SEQ |
|---|---|---|---|
| Gene | Accession | | ID |
| Symbol | Number | Target Sequence | NO |
| TIMP1 | NM_011593.2 | AAGCCTCTGTGGATATGCCCACAA | 1 |
| | | GTCCCAGAACCGCAGTGAAGAGTT | |
| | | TCTCATCACGGGCCGCCTAAGGAA | |
| | | CGGAAATTTGCACATCAGTGCCTG | |
| | | CAGC | |
| COL1A1 | NM_007742.3 | CAATGGTGAGACGTGGAAACCCG | 2 |
| | | AGGTATGCTTGATCTGTATCTGCC | |
| | | ACAATGGCACGGCTGTGTGCGAT | |
| | | GACGTGCAATGCAATGAAGAACT | |
| | | GGACTGT | |
| CYP7A1 | NM_007824.2 | CTCTCTGAAGCCATGATGCAAAAC | 3 |
| | | CTCCAATCTGTCATGAGACCTCCG | |
| | | GGCCTTCCTAAATCAAAGAGCGCT | |
| | | GTCTGGGTCACGGAAGGGATGTAT | |
| | | GCCT | |
| B2M | NM_009735.3 | CATACGCCTGCAGAGTTAAGCAT | 4 |
| | | GCCAGTATGGCCGAGCCCAAGAC | |
| | | CGTCTACTGGGATCGAGACATGT | |
| | | GATCAAGCATCATGATGCTCTGAA | |
| | | GATTCAT | |
| HPRT | NM_013556.2 | TGCTGAGGCGGCGAGGGAGAGC | 5 |
| | | GTTGGGCTTACCTCACTGCTTTCC | |
| | | GGAGCGGTAGCACCTCCTCCGCC | |
| | | GGCTTCCTCCTCAGACCGCTTTTT | |
| | | GCCGCGA | |
| PGK1 | NM_008828.2 | CCGGCATTCTGCACGCTTCAAAAG | 6 |
| | | CGCACGTCTGCCGCGCTGTTCTCC | |
| | | TCTTCCTCATCTCCGGGCCTTTCGA | |
| | | CCTCACGGTGTTGCCAAAATGTCG | |
| | | CTT | |
| RPL13a | NM_009438.5 | ATGGGATCCCTCCACCCTATGAC | 7 |
| | | AAGAAAAAGCGGATGGTGGTCCC | |
| | | TGCTGCTCTCAAGGTTGTTCGGC | |
| | | TGAAGCCTACCAGAAAGTTTGCTT | |
| | | ACCTGGG | |
| RPN1 | NM_133933.3 | GGCAGCCTGACAGTGGGATCTCC | 8 |
| | | TCCATTCGTTCTTTTAAGACCATC | |
| | | CTTCCTGCTGCCGCCCAGGATGT | |
| | | CTATTACCGGGATGAGATTGGTAA | |
| | | TGTTTC | |

### Results

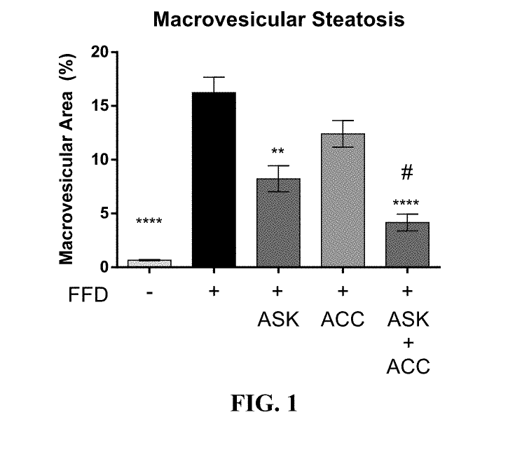

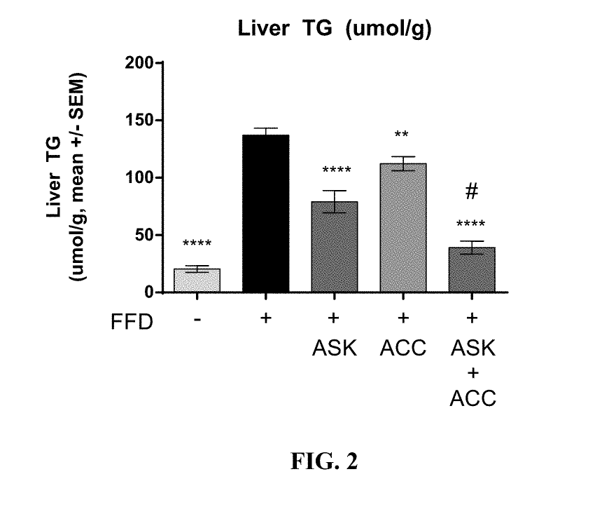

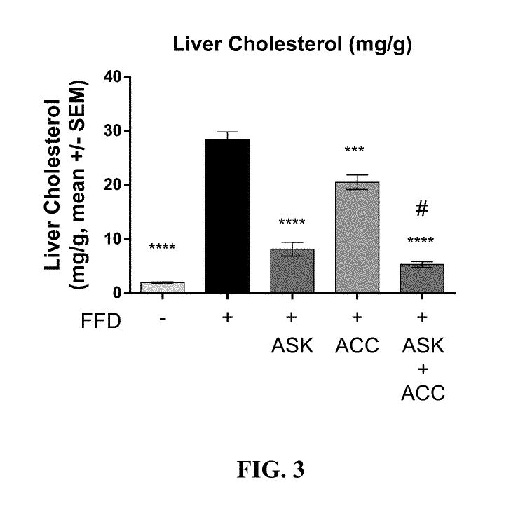

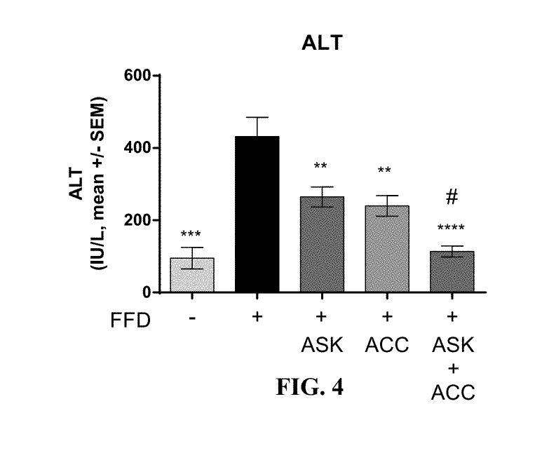

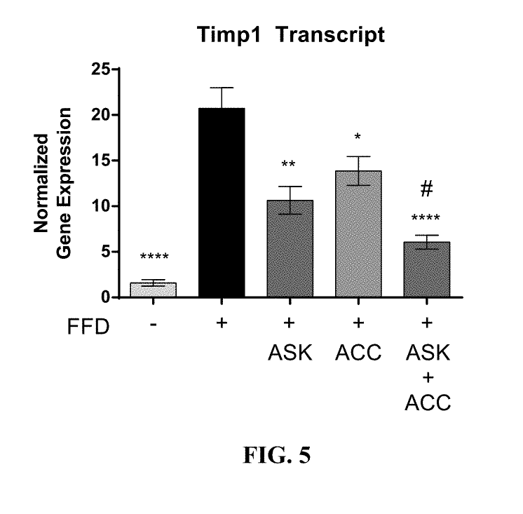

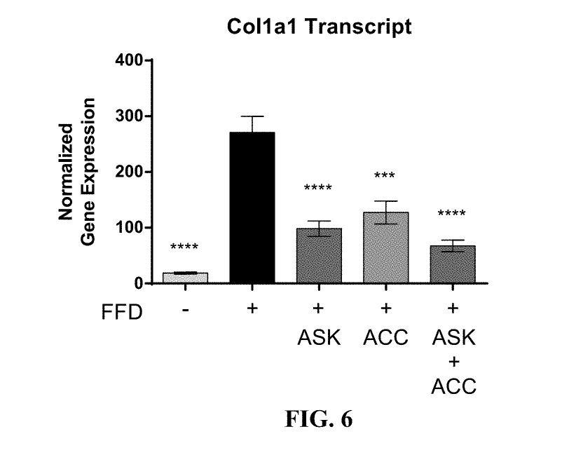

Example 1 indicates that a combined treatment with an ASK1 inhibitor and an ACC inhibitor results in greater anti-steatoic efficacy than either agent administered alone. In particular FIG. 1 shows a significant reduction in macrovesicular steatosis for the combination of the compound of Formula (I) and the compound of Formula (III). Example 1 also shows significant improvement for the combination for liver triglycerides (FIG. 2), liver cholesterol (FIG. 3), and serum ALT (FIG. 4) for the combination of the compound of Formula (I) and the compound of Formula (III). Additionally the combination of the compound of Formula (I) and the compound of Formula (III) shows a trend toward reduction of the pro-fibrotic transcript CollA1 (FIG. 6) and the combination showed a significant reduction in Timp1 (FIG. 5) transcript.

### Example 2. Efficacy in Choline-Deficient, High Fat, High Cholesterol NASH Model

The following study was conducted to evaluate the efficacy of the combination of an ASK1 inhibitor (ASK1i) and an ACC inhibitor (ACCi) in a rodent model of non-alcoholic steatohepatitis (NASH) relative to the efficacy of the individual agents alone in the model. In this model, NASH was induced in male Wistar rats by administration of a choline-deficient high fat diet (CDHFD).

### Animals

Male Wistar (Crl:Wi(Han)) rats (aged 8-9 weeks at arrival) were acquired from Charles River, Raleigh, N.C. and used in the current studies. This study complie with all applicable sections of the Final Rules of the Animal Welfare Act regulations (Code of Federal Regulations, Title 9), the Public Health Service Policy on Humane Care and Use of Laboratory Animals from the Office of Laboratory Animal Welfare, and the Guide for the Care and Use of Laboratory Animals from the National Research Council.

### Vehicle Preparation

The vehicle, w/v 50 mM tris buffer, pH 8 in deionized water, was prepared prior to use and stored in a refrigerator set to maintain 2-8° C. To prepare 1 L, 800 mL of hot water (˜80° C.) was added to an appropriate container and stirred vigorously until a steep vortex formed. 5.0 grams of sodium methylcellulose was slowly added to the sodium carboxymethylcellulose to the vortex. Stirring was continued until all carboxymethylcellulose was dissolved and the solution cooled down to ambient temperature. 5.12 g of Tris HCl was added to the container. 2.12 grams of Tris base was added to the container. 10 g of ethanol was added to the container. The components were stirred for approximately 15 minutes, ensuring all solids have dissolved. QS water was added to 1 L with gentle mixing.

Formula (III) Preparation

Formula (III) formulations were prepared using the vehicle by diluting Formula (III) to the desired concentration.

Study Design

Food was pro libitum and all animals on study were given a choline-deficient, high fat, high cholesterol diet (CDHFD; Research Diets, A16092003) on Day 1 of study except for group 1, the control chow group, which received standard diet (5CR4), as outlined in Table 3. For those animals receiving the ASK1 inhibitor, ASKli was administered in the diet (Diet A16111101). Diet A16111101 is a choline-deficient, high fat, high cholesterol diet that has had ASKli added (0.03%). On the day of sacrifice, Liver was harvested and paraffin embedded, and plasma was collected and frozen. Animals were not dosed the day of sacrifice.

TABLE 3
Experimental Design and Dose Groups

| Group | Group name | n | Diet (weeks) | Treatment (PO) |
|---|---|---|---|---|
| 1 | Control | 10 | Standard Diet (0-12) | N/A |
| 2 | Start of | 10 | CDHFD (0-6) | N/A |
| | Treatment | | | |
| 3 | Vehicle | 15 | CDHFD (1-12) | N/A |
| 4 | ASK1i | 15 | CDHFD (1-12) | 0.03% ASK1i in chow |
| 5 | ACCi | 15 | CDHFD (1-12) | 10 mg/kg QD |
| | | | | Formula (III) |
| 6 | ASK1i + | 15 | CDHFD (1-12) | 10 mg/kg QD |
| | ACCi | | | Formula (III) |
| | | | | 0.03% ASK1i in chow |

Tissues were collected by Charles River in Reno, Nev., processed and embedded in paraffin at Histo-tec in Hayward, Calif. and then shipped to Gilead Sciences in Foster City. 5 μm thick tissue sections were prepared for staining.

Picrosirius Red Staining:

Sections were pretreated in 0.2% Phosphomolybdic Acid (EMS, Cat#26357-01) and then subsequently incubated in 0.1% (W/V) Sirius Red 88-89-1 in saturated Picric acid solution (EMS, Cat#26357-02) for 1 hour at room temperature. This was followed by differentiation in 0.01N HCl (EMS, Cat#26357) and dehydration in graded alcohols.

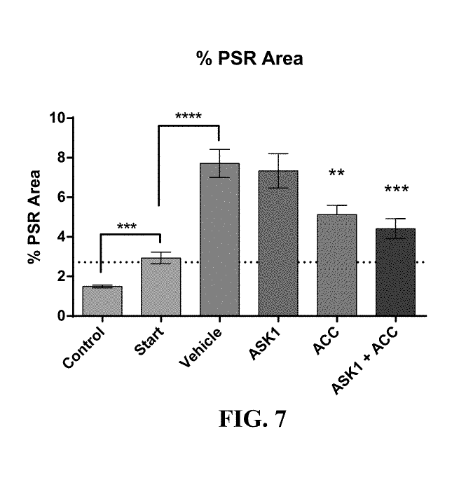

Whole slide images of Picrosirius Red (PSR) stained slides were captured using a Leica AT2 scanner at 40× magnification. Digital slide images were checked for scanning quality, annotated and exported to appropriate network folders within Leica Digital Image Hub archive. Quantitative image analysis was performed on the whole slide images using Visiopharm image analysis software (Visiopharm, Hoersholm, Denmark) to determine the extent and intensity of PSR. The total PSR-stained area was measured and expressed as a percentage of total liver area stained. Results are shown in FIG. 7.

Plasma TIMP-1 ELISA:

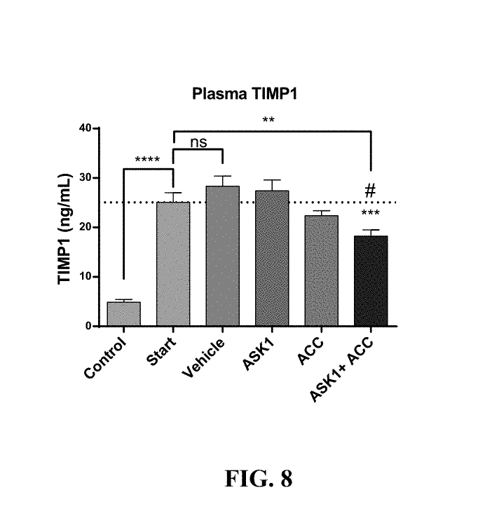

Plasma TIMP-1 concentrations were determined in duplicate using a commercially available rat TIMP-1 specific ELISA kit (R&D Systems, Minneapolis, Minn.). TIMP-1 was assayed in plasma according to the manufacturer's specifications with minor modifications. Buffer RD1-21 (50 μL) was added to ELISA plate wells pre-coated with mouse anti-TIMP-1. Prior to ELISA, a seven point standard curve of rat TIMP-1 (NSO-expressed recombinant TIMP-1: 2400-37.5 pg/mL) was generated and plasma samples were diluted 1:100 in buffer RD5-17. Samples and standards (50 μL each) were added in duplicate to wells containing RD1-21 and incubated (room temperature) for 2 hours on an orbital plate shaker (300 rpm). Following antigen capture, plates were washed 5 times (350 μL/well/wash) with Wash Buffer using an automated plate washer. Following washing, rat TIMP-1 conjugate (100 μl) was added to each well and plates were incubated (room temperature) for 2 hours on an orbital plate shaker (300 rpm). Plates were then washed 5 times and Substrate Solution (100 μL) was added to each well. Plates were incubated at room temperature for 30 minutes protected from light. Finally, Stop Solution (100 μL) was added to each well. Optical Density (O.D.) absorbance was immediately determined at 450 nm on a SpectraMax 190 microplate reader (Molecular Devices, Sunnyvale Calif.). Relative O.D.s for each standard and sample were background corrected against blank samples, and standard curves for conversion of O.D.s to TIMP-1 concentration were generated using a 4 Parameter curve fit method. Unknown sample TIMP-1 concentrations were determined using SoftMax ProS software using a dilution factor of 100. Results are shown in FIG. 8.

Plasma Hyaluronic Acid (HA) Assay:

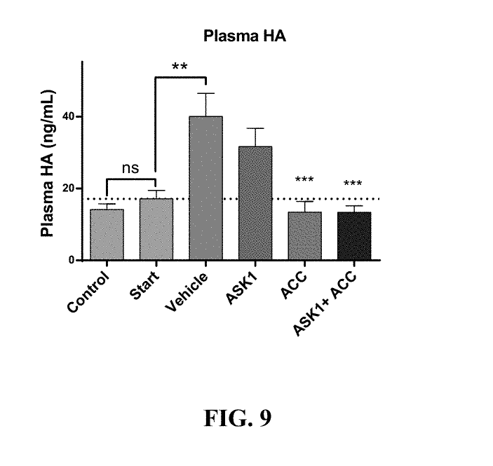

Plasma HA concentrations were determined in duplicate using a commercially available HA Test Kit (Corgenix, Inc., Broomfield, Colo.). HA was assayed in plasma according to the manufacturer's specifications with minor modifications. Prior to assay, a seven point standard curve of HA reference solution (800-12.5 ng/mL) was generated and each reference sample and plasma sample was diluted 1 part to 10 parts Reaction Buffer (30 μl reference/sample to 300 μL Reaction Buffer). Samples and standards (100 μl) were added in duplicate to microplate wells pre-coated with HA binding protein (HABP) and incubated (room temperature) for 60 minutes on an orbital plate shaker (300 rpm). Following antigen capture, plates were washed 4 times (350 μL/well/wash) with PBS using an automated plate washer. Following washing, HRP-conjugated HABP (100 μL) was added to each well and plates were incubated (room temperature) for 30 minutes on an orbital plate shaker (300 rpm). Plates were then washed 4 times and the one-component Substrate Solution (100 μl) was added to each well. Plates were incubated at room temperature for 30 minutes protected from light. Finally, Stop Solution (100 μl) was added to each well. Optical Density (O.D.) absorbance was immediately determined at 450 nm on a SpectraMax 190 microplate reader (Molecular Devices, Sunnyvale Calif.). Relative O.D.s for each standard and sample were background corrected against blank samples, and standard curves for conversion of O.D.s to HA concentration were generated using a 4 Parameter curve fit method. Unknown sample HA concentrations were determined using SoftMax ProS software. Results are shown in FIG. 9.

### Results

Example 2 demonstrates the anti-fibrotic efficacy of ASK1i and ACCi. CDHFD significantly increased hepatic PSR at 6 (2.7% area) and at 12 weeks (8.3% area) compared to rats on normal diet. Treatment with ASK1i, ACCi, or ASK1i+ ACCi reduced PSR by 18% (ns), 50% (p<0.05), and 59% (p<0.01), respectively. Plasma levels of TIMP1 were increased in CDHFD rats and were reduced below start of treatment levels by ASK1i+ ACCi (p<0.05). HA was reduced in all ACCi-containing groups.

### Example 3. Pre-Clinical Study of Apoptosis-Signal Regulating Kinase (ASK1) Inhibitor (Formula (II)) in Combination with an Acetyl-CoA Carboxylase Inhibitor (Formula (IV)) in NASH

70 subjects with NASH diagnosed by a hepatic proton density fat fraction (PDFF) ≥10% and liver stiffness ≥2.88 kPa by MRE, or liver biopsy consistent with NASH and stage 2-3 fibrosis were enrolled. Successive cohorts received monotherapy with Formula (II) 18 mg, Formula (IV) 20 mg, or combination therapy with Formula (II)+Formula (IV) (18/20 mg) orally QD for 12 weeks. Centrally-read PDFF and MRE, and serum fibrosis markers were measured at baseline (BL), W4 and W12. Deuterated water was administered to measure fractional synthesis of lipids (de novo lipogenesis [DNL]).

Over 12 weeks, all regimens were safe and well-tolerated. Similar rates of AEs were observed between monotherapy and combination cohorts (Table 4). No subject discontinued treatment prematurely. Compared with BL, Formula (IV) resulted in significant improvements in PDFF (p=0.006) and TIMP-1 (p=0.049), and non-significant reductions in ALT and PIII-NP (Table 4). The combination of Formula (II)+Formula (IV) led to significant reductions in PDFF (p<0.001), ALT (p=0.019), and PIII-NP (p=0.057).

TABLE 4
Safety and Relative (%) Changes in Imaging, Liver
Biochemistry, and Serum Fibrosis Markers at W 12 †

| | | | | | Formula (II) + | |
|---|---|---|---|---|---|---|
| | Formula (II) | | Formula (IV) | | Formula (IV) | |
| | 18 mg (n = 10) | | 20 mg (n = 10) | | 18/20 mg (n = 20) | |
| MRI-PDFF | 7.1 | (−16.3, 28.9) | −42.7* | (−52.3, −19.4) | −32.0* | (−45.3, −2.6) |
| ≥30% reduction in MRI-PDFF | 10% | (1) | 70% | (7) | 50% | (10) |
| MRE | −8.6 | (−15.6, 13.6) | −8.9 | (−15.1, −6.3) | −4.5 | (−17.7, 9.3) |
| ALT | −1.2 | (−24.0, 11.4) | −33.5 | (−39.8, −17.9) | −27.2* | (−42.8, −10.4) |
| GGT | −4.4 | (−17.3, 6.5) | −1.6 | (−19.5, 11.5) | 10.1 | (−21.5, 19.2) |
| TIMP-1 | 2.6 | (−4.0, 16.7) | −11.6* | (−17.1, 1.8) | −1.9 | (−11.3, 11.3) |
| PIII-NP | −8.7 | (−20.3, 15.4) | −11.9 | (−29.1, 22.2) | −11.4 | (−25.4, −2.9) |
| Grade 2 or higher AE | 40% | (4) | 40% | (4) | 25% | (5) |

† All data are median (IQR) relative (%) changes from BL, or % (n).
*p < 0.05 vs. BL.

In this study in patients with NASH, 12-week treatment with the combination of Formula (II)+Formula (IV) was safe and led to improvements in hepatic steatosis, liver biochemistry, and fibrosis markers.

1. A method of treating and/or preventing a liver disease in a patient in need thereof, comprising administering to the patient a therapeutically effective amount of an ASK1 inhibitor in combination with a therapeutically effective amount of an ACC inhibitor, wherein the ASK1 inhibitor is a compound of Formula (I): 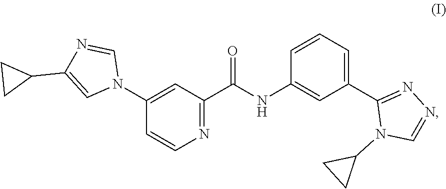 [SMILES: O=C(Nc1cccc(-c2nncn2C2CC2)c1)c1cc(-n2cnc(C3CC3)c2)ccn1]
or a pharmaceutically acceptable salt thereof;
and the ACC inhibitor is a compound of Formula (III): 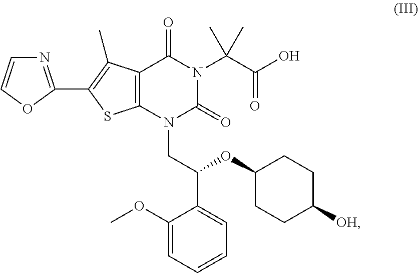 [SMILES: COc1ccccc1[C@H](Cn1c(=O)n(C(C)(C)C(=O)O)c(=O)c2c(C)c(-c3ncco3)sc21)O[C@H]1CC[C@@H](O)CC1]
or a pharmaceutically acceptable salt thereof.

2. A method of treating and/or preventing a liver disease in a patient in need thereof, comprising administering to the patient a therapeutically effective amount of an ASK1 inhibitor in combination with a therapeutically effective amount of an ACC inhibitor, wherein the ASK1 inhibitor is a compound of Formula (I): 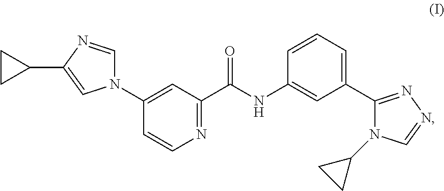 [SMILES: O=C(Nc1cccc(-c2nncn2C2CC2)c1)c1cc(-n2cnc(C3CC3)c2)ccn1]
or a pharmaceutically acceptable salt thereof;
and the ACC inhibitor is a compound of Formula (IV): 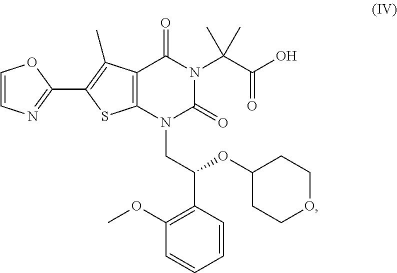 [SMILES: COc1ccccc1[C@H](Cn1c(=O)n(C(C)(C)C(=O)O)c(=O)c2c(C)c(-c3ncco3)sc21)OC1CCOCC1]
or a pharmaceutically acceptable salt thereof.

3. A method of treating and/or preventing a liver disease in a patient in need thereof, comprising administering to the patient a therapeutically effective amount of an ASK1 inhibitor in combination with a therapeutically effective amount of ACC inhibitor, wherein the ASK1 inhibitor is a compound of Formula (II): 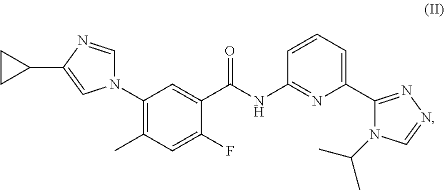 [SMILES: Cc1cc(F)c(C(=O)Nc2cccc(-c3nncn3C(C)C)n2)cc1-n1cnc(C2CC2)c1]
or a pharmaceutically acceptable salt thereof;
and the ACC inhibitor is a compound of Formula (III): 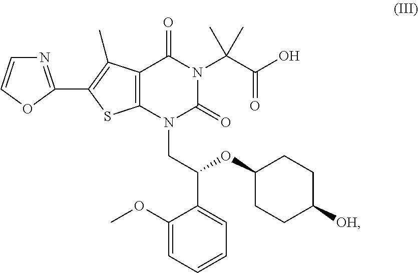 [SMILES: COc1ccccc1[C@H](Cn1c(=O)n(C(C)(C)C(=O)O)c(=O)c2c(C)c(-c3ncco3)sc21)O[C@H]1CC[C@@H](O)CC1]
or a pharmaceutically acceptable salt thereof.

4. A method of treating and/or preventing a liver disease in a patient in need thereof, comprising administering to the patient a therapeutically effective amount of an ASK1 inhibitor in combination with a therapeutically effective amount of ACC inhibitor, wherein the ASK1 inhibitor is a compound of Formula (II): 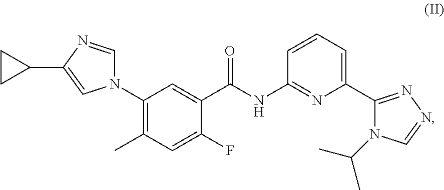 [SMILES: Cc1cc(F)c(C(=O)Nc2cccc(-c3nncn3C(C)C)n2)cc1-n1cnc(C2CC2)c1]
or a pharmaceutically acceptable salt thereof;
and the ACC inhibitor is a compound of Formula (IV): 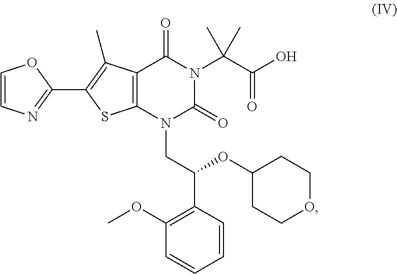 [SMILES: COc1ccccc1[C@H](Cn1c(=O)n(C(C)(C)C(=O)O)c(=O)c2c(C)c(-c3ncco3)sc21)OC1CCOCC1]
or a pharmaceutically acceptable salt thereof.

5. The method of claim 1, wherein the ASK1 inhibitor and the ACC inhibitor are administered together.

6. The method of claim 1, wherein the ASK1 inhibitor and the ACC inhibitor are administered separately.

7. The method of claim 1, wherein the liver disease is non-alcoholic steatohepatitis (NASH).

8. A pharmaceutical composition comprising a therapeutically effective amount of an ASK1 inhibitor and a therapeutically effective amount of an ACC inhibitor, wherein the ASK1 inhibitor is a compound of Formula (I): 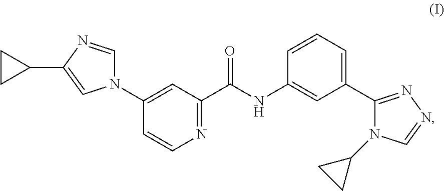 [SMILES: O=C(Nc1cccc(-c2nncn2C2CC2)c1)c1cc(-n2cnc(C3CC3)c2)ccn1]
or a pharmaceutically acceptable salt thereof;
and the ACC inhibitor is a compound of Formula (III): 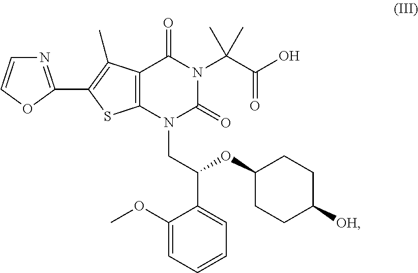 [SMILES: COc1ccccc1[C@H](Cn1c(=O)n(C(C)(C)C(=O)O)c(=O)c2c(C)c(-c3ncco3)sc21)O[C@H]1CC[C@@H](O)CC1]
or a pharmaceutically acceptable salt thereof.

9. A pharmaceutical composition comprising a therapeutically effective amount of an ASK1 inhibitor and a therapeutically effective amount of an ACC inhibitor, wherein the ASK1 inhibitor is a compound of Formula (I): 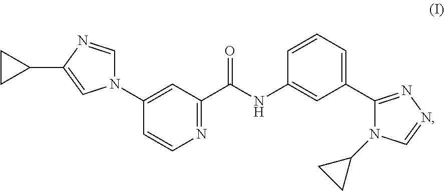 [SMILES: O=C(Nc1cccc(-c2nncn2C2CC2)c1)c1cc(-n2cnc(C3CC3)c2)ccn1]
or a pharmaceutically acceptable salt thereof;
and the ACC inhibitor is a compound of Formula (IV): 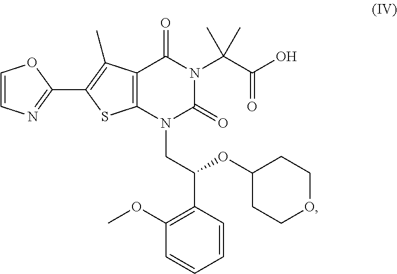 [SMILES: COc1ccccc1[C@H](Cn1c(=O)n(C(C)(C)C(=O)O)c(=O)c2c(C)c(-c3ncco3)sc21)OC1CCOCC1]
or a pharmaceutically acceptable salt thereof.

10. A pharmaceutical composition comprising a therapeutically effective amount of an ASK1 inhibitor and a therapeutically effective amount of an ACC inhibitor, wherein the ASK1 inhibitor is a compound of Formula (II): 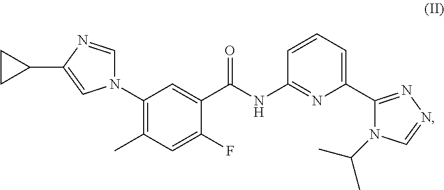 [SMILES: Cc1cc(F)c(C(=O)Nc2cccc(-c3nncn3C(C)C)n2)cc1-n1cnc(C2CC2)c1]
or a pharmaceutically acceptable salt thereof;
and the ACC inhibitor is a compound of Formula (III): 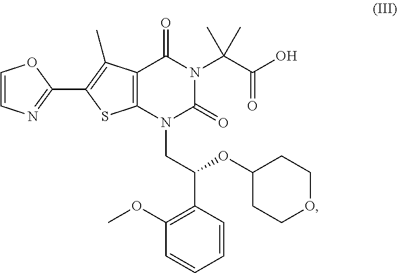 [SMILES: COc1ccccc1[C@H](Cn1c(=O)n(C(C)(C)C(=O)O)c(=O)c2c(C)c(-c3ncco3)sc21)OC1CCOCC1]
or a pharmaceutically acceptable salt thereof.

11. A pharmaceutical composition comprising a therapeutically effective amount of an ASK1 inhibitor and a therapeutically effective amount of an ACC inhibitor, wherein the ASK1 inhibitor is a compound of Formula (II): 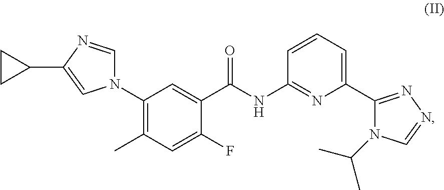 [SMILES: Cc1cc(F)c(C(=O)Nc2cccc(-c3nncn3C(C)C)n2)cc1-n1cnc(C2CC2)c1]
or a pharmaceutically acceptable salt thereof;
and the ACC inhibitor is a compound of Formula (IV): 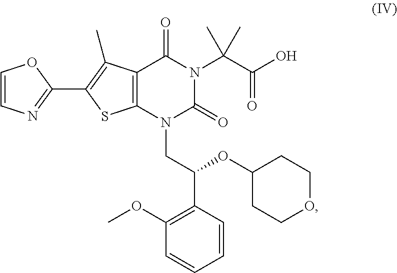 [SMILES: COc1ccccc1[C@H](Cn1c(=O)n(C(C)(C)C(=O)O)c(=O)c2c(C)c(-c3ncco3)sc21)OC1CCOCC1]
or a pharmaceutically acceptable salt thereof.

12. The pharmaceutical composition of claim 8, further comprising a pharmaceutically acceptable carrier.


SEQUENCE LISTING (8 sequences)

SEQ ID NO: 1 - DNA, 100 bp, Artificial Sequence
  Synthetic
aagcctctgtggatatgcccacaagtcccagaaccgcagtgaagagtttctcatcacgggccgcctaaggaacggaaatttgcacatcagtgcctgcagc

SEQ ID NO: 2 - DNA, 100 bp, Artificial Sequence
  Synthetic
caatggtgagacgtggaaacccgaggtatgcttgatctgtatctgccacaatggcacggctgtgtgcgatgacgtgcaatgcaatgaagaactggactgt

SEQ ID NO: 3 - DNA, 100 bp, Artificial Sequence
  Synthetic
ctctctgaagccatgatgcaaaacctccaatctgtcatgagacctccgggccttcctaaatcaaagagcgctgtctgggtcacggaagggatgtatgcct

SEQ ID NO: 4 - DNA, 100 bp, Artificial Sequence
  Synthetic
catacgcctgcagagttaagcatgccagtatggccgagcccaagaccgtctactgggatcgagacatgtgatcaagcatcatgatgctctgaagattcat

SEQ ID NO: 5 - DNA, 100 bp, Artificial Sequence
  Synthetic
tgctgaggcggcgagggagagcgttgggcttacctcactgctttccggagcggtagcacctcctccgccggcttcctcctcagaccgctttttgccgcga

SEQ ID NO: 6 - DNA, 100 bp, Artificial Sequence
  Synthetic
ccggcattctgcacgcttcaaaagcgcacgtctgccgcgctgttctcctcttcctcatctccgggcctttcgacctcacggtgttgccaaaatgtcgctt

SEQ ID NO: 7 - DNA, 100 bp, Artificial Sequence
  Synthetic
atgggatccctccaccctatgacaagaaaaagcggatggtggtccctgctgctctcaaggttgttcggctgaagcctaccagaaagtttgcttacctggg

SEQ ID NO: 8 - DNA, 100 bp, Artificial Sequence
  Synthetic
ggcagcctgacagtgggatctcctccattcgttcttttaagaccatccttcctgctgccgcccaggatgtctattaccgggatgagattggtaatgtttc